(19) United States
(12) Patent Application Publication
(10) Pub. No.: US 2003/0175737 A1
(43) Pub. Date: Sep. 18, 2003

(54) Quantifying target molecules contained in a liquid

(76) Inventors: Jurgen Schulein, Spardorf (DE); Bjorn Grassl, Nurnberg (DE); Jorg Hassmann, Erlangen (DE)

Correspondence Address: Mark S Ellinger, Fish & Richardson, Suite 3300, 60 South Sixth Street, Minneapolis, MN, 55402, US

(21) Appl. No.: 10/220,401

(22) PCT Filed: Feb. 28, 2001
(86) PCT No.: PCT/DE01/00736

(30) Foreign Application Priority Data
Mar. 1, 2000 (DE) 100 09 715.4

(51) Int. Cl. C12Q 1/68; G01N 27/26

(52) U.S. Cl. 435/6; 205/777.5

(57) ABSTRACT

The invention relates to a method for detecting and/or quantifying first biopolymers contained in a liquid involving the following steps: a) providing an electrode with a surface which is made of plastic and which is coated with second biopolymers that have a specific affinity to the first biopoymers to be detected; b) bringing the electrode into contact with the liquid; c) applying a predetermined voltage protocol to the electrode in order to effect a concentration of the first biopolymers on the second biopolymers; d) adding osmium tetroxide and bipyridine to the liquid, and; e) measuring the redox signal coming off the electrode.

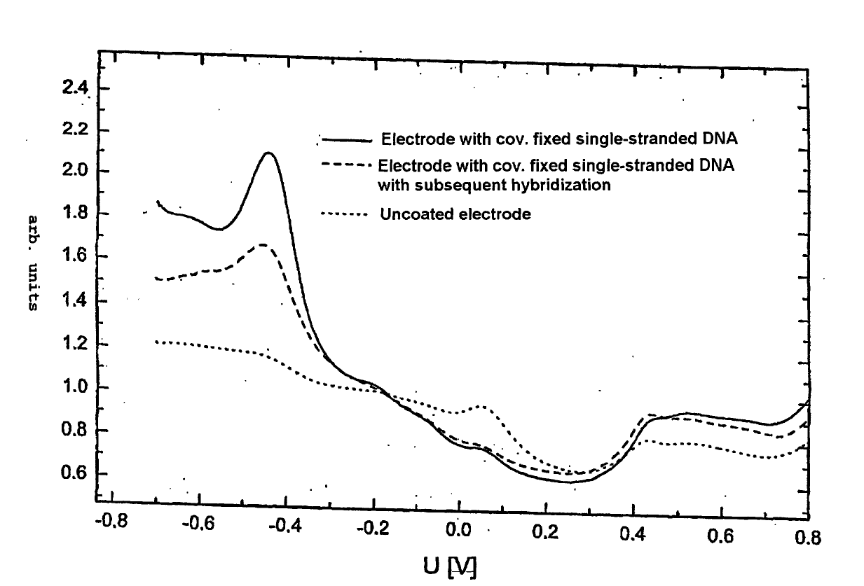

The invention relates to a process for the detection and/or quantification of target molecules present in a liquid.

In accordance with the prior art, WO 96/01836 discloses a chip for the detection of polynucleotide sequences. A multiplicity of miniaturized reaction fields is provided on the chip, which is made from a silicon substrate. A probe is connected to each of the reaction fields. On immersion of the chip into a solution containing the polynucleotide sequence to be detected, hybridization with one of the probes provided occurs. The hybridization can be detected, for example, by fluorophoric labeling provided on the probe.

DE 198 08 884.1 describes a process for the detection of chemical substances using two interacting fluorophoric groups which are bonded to a molecule. In the case of specific adduction of the molecule onto the chemical substance to be detected, the interaction between the fluorophoric groups is modified.

WO 99/47700 relates to a process for the detection of a target molecule by means of fluorescence. In this process, a probe provided with a fluorophoric group is bound to a solid phase. In the presence of the target sequence in the solution, a second fluorophoric group is bound in the vicinity of the first fluorophoric groups in such a way that radiation-free energy transfer between the two fluorophoric groups can occur.

U.S. Pat. Nos. 5,312,572 and 5,871,918 describe processes for the electrochemical detection of polynucleotide sequences. In these processes, redox-active molecules which, on hybridization of the polynucleotide sequence, bind to the double-stranded molecule formed are added to the solution. The presence of a double-stranded molecule of this type causes a measurable redox signal.

U.S. Pat. No. 5,591,578 describes a process for the detection of polynucleotide sequences using redox indicators. In this process, a probe which is complementary to the target polynucleotide sequence is covalently bonded to an electrode. Redox-active transition-metal complexes are covalently bonded to the probe. On hybridization of the target polynucleotide sequence with the probe, a redox signal can be measured at the electrode.

DE 196 28 171 discloses a process for the purification and enrichment of charge-carrying first molecules which have a specific affinity to second molecules bonded to an electrode. When a solution containing the first molecules is brought into contact with the electrode, a voltage program is run through in such a way that the first molecules are enriched at the electrode.

E. Palecek, Bioelectrochemistry and Bioenergetics 1985, 15, 275-295, discloses the use of osmium tetroxide compounds as redox-active substance for the detection of double-stranded biopolymers.

The processes disclosed in the prior art are time-consuming, inconvenient or require complex equipment.

The object of the invention is to overcome the disadvantages of the prior art. In particular, the aim is to indicate a sensitive, simple and inexpensive electrochemical process for the detection and/or quantification of small amounts of first biopolymers present in a liquid.

This object is achieved by the features of claim 1. Advantageous embodiments arise from the features of claims 2-12.

In accordance with the invention, provision is made for a process for the detection and/or quantification of first biopolymers present in a liquid, having the following steps:

a) provision of an electrode having a surface made of plastic which is coated with second biopolymers which have a specific affinity to the first biopolymers to be detected,

b) bringing of the electrode into contact with the liquid,

c) application of a pre-specified voltage program to the electrode, causing enrichment of the first biopolymers at the second biopolymers,

d) addition of osmium tetroxide and bipyridine to the liquid,

e) measurement of the redox signal falling off at the electrode.

The proposed process enables sensitive detection of first biopolymers present in a liquid. The use of electrodes provided with a plastic surface enables the process to be carried out inexpensively. In particular, the process also enables quantification of the first biopolymers present in the liquid.

The term first and second biopolymers here is taken to mean, in particular, proteins, peptides, DNA, RNA and the like. The first biopolymer may be, in particular, a single-stranded DNA or RNA which is complementary to the second biopolymer.

The second biopolymers are preferably covalently bonded to the plastic surface. In combination with the proposed use of osmium tetroxide and bipyridine, particularly high sensitivity is achieved.

According to an advantageous embodiment, the plastic is an electrically conductive composite material, for example a composite of carbon fibers and polycarbonate. The electrode advantageously consists entirely of the plastic. Such electrodes can be produced in an inexpensive pressing process.

It is furthermore possible for the second biopolymers to be bonded to a matrix, preferably made of dextran or polyethylene glycol, applied to the surface of the electrode. The use of a matrix of this type enables the coverage density of the surface of the electrode with second biopolymers to be increased.

According to a further embodiment, one of the following measurements is carried out in step e: direct-voltage measurement, cyclovoltammetric measurement, chronoamperometric measurement, chronovoltammetric measurement. Furthermore, a differential pulse voltammogram or an impendance spectrum can be recorded in step e. It is also possible to measure an alternating-current signal phase-sensitively in step e. A direct-voltage signal may be superimposed on the alternating-current signal. In order to quantify the first biopolymers, integration can be carried out via a peak of the measurement signal. The quantification parameter that can be utilized is the separation between peak height and background.

In order to carry out multiple measurements, the electrode can be rinsed or heated after step e. Heating of the electrode facilitates thermal denaturing of the first biopolymers. Certain first biopolymers preferentially bind at a pre-specified temperature. Heating or setting of the temperature enables the specificity of the process to be increased further. The specificity or stringency can also be increased by suitable setting of the pH in the liquid.

The first biopolymers are advantageously subjected to a polymerase chain reaction before step a. This enables the detection of particularly small amounts of first biopolymers.

The process is explained in greater detail with reference to the drawing and a working example.

The single FIGURE shows a differential pulse voltammogram of an uncoated working electrode, a working electrode coated with single-stranded oligonucleotides and a working electrode coated with a hybridized oligonucleotide, in each case after treatment with osmium tetroxide and bipyridine. The working electrodes each consist of carbon composite material, which is preferably composed of 30% of carbon fibers and 70% of polycarbonate. Oligonucleotides containing the sequence 5′-GCC TTC CCA ACC ATT CCC TTA-3′ were covalently bonded to the surface of the working electrodes using carbodiimide by a standard method. The coverage density was 15 fmol/mm². The hybridization of the oligonucleotides was carried out in a buffered solution of 0.5-fold TBE (TRIS borate EDTA) 0.5 M NaCl and 100 fmol/μl of complementary oligonucleotides. After the hybridization, the working electrodes were washed stringently.

An untreated working electrode, a working electrode coated with single-stranded oligonucleotides and a working electrode coated with hybridized oligonucleotides were subsequently each dipped in a solution of 2 mM OSO₄ and 13 mM bipyridine for 30 seconds. The measurement was carried out with the aid of a platinum counterelectrode and an Ag/AgCl reference electrode using an Ecochemie PGSTAT 10 Autolab.

The hybridization of the target oligonucleotides at the working electrode can be accelerated by application of a voltage. The coverage density of oligonucleotides on the surface of the working electrode can be increased by addition of salt during the coating or by basic pretreatment of the surface. For example, a coverage density of 85 fmol/mm² can be achieved in a 10 mM MgCl₂ solution. In the case of pretreatment of the surface for three hours in 5 M NaOH, a coverage density of 750 fmol/mm² can be achieved.

If a plurality of measurements are to be carried out one after the other, an opposite voltage can be applied to the electrode after step d. In addition, the electrode can be rinsed and/or heated after step e. The heating of the electrode facilitates thermal denaturing of the first biopolymers. However, heating or setting of the temperature of the electrode also enables the specificity of the process to be increased since prespecified first biopolymers bind at a specific temperature.

1. A process for the detection and/or quantification of first biopolymers present in a liquid, having the following steps:
a) provision of an electrode having a surface made of plastic which is coated with second biopolymers which have a specific affinity to the first biopolymers to be detected,
b) bringing of the electrode into contact with the liquid,
c) application of a pre-specified voltage program to the electrode, causing enrichment of the first biopolymers at the second biopolymers,
d) addition of osmium tetroxide and bipyridine to the liquid,
e) measurement of the redox signal falling off at the electrode.

2. A process as claimed in claim 1, in which the plastic is an electrically conductive composite material.

3. A process as claimed in one of the preceding claims, in which the electrode consists entirely of plastic.

4. A process as claimed in one of the preceding claims, in which the second biopolymers are bonded to a matrix, preferably made of dextran or polyethylene glycol, applied to the surface of the electrode.

5. A process as claimed in one of the preceding claims, in which one of the following measurements is carried out in step e: direct-voltage measurement, cyclovoltammetric measurement, chronoamperometric measurement, chronovoltammetric measurement.

6. A process as claimed one of claims 1 to 3, in which a differential pulse voltammogram is recorded in step e.

7. A process as claimed in one of claims 1 to 3, in which an impendance spectrum is recorded in step e.

8. A process as claimed in one of claims 1 to 3, in which an alternating-current signal is measured phase-sensitively in step e.

9. A process as claimed in claim 5, in which the alternating-current signal is superimposed on a direct-voltage signal.

10. A process as claimed in one of the preceding claims, in which, in order to quantify the first biopolymers, integration is carried out via a peak of a measurement signal.

11. A process as claimed in one of the preceding claims, in which, for multiple measurement, the electrode is rinsed or heated after step e.

12. A process as claimed in one of the preceding claims, in which the first biopolymers are subjected to a polymerase chain reaction before step a.
